(19) United States
(12) Patent Application Publication
(10) Pub. No.: US 2011/0104735 A1
(43) Pub. Date: May 5, 2011

(54) HUMAN OMENTAL MESOTHELIAL CELLS, METHODS OF ISOLATION AND USES THEREOF

(75) Inventors: BENJAMIN MARCUS BUEHRER, CHAPEL HILL, NC (US); RICHARD BENTLEY CHEATHAM, DURHAM, NC (US); JAMES BRADFORD NICOLL, RALEIGH, NC (US); PETER EARL PIERACCINI, DURHAM, NC (US)

(73) Assignee: ZENBIO, INC.

(21) Appl. No.: 12/916,932

(22) Filed: Nov. 1, 2010

Related U.S. Application Data
(60) Provisional application No. 61/256,770, filed on Nov. 1, 2009.
(60) Provisional application No. 61/259,316, filed on Nov. 9, 2009.

(51) Int. Cl. C12Q 1/02; C12N 5/071; G01N 33/53

(52) U.S. Cl. 435/29; 435/366; 435/369; 435/370; 435/371; 436/86; 435/378; 435/380

(57) ABSTRACT

The present invention discloses novel methods and omental, myocardial, liver, lung, renal, peritoneal, intestinal and pancreatic mesothelial cells which are useful for a number of procedures including drug discovery, co-culturing, cell therapy and bioassay. The invention provides a method for isolating these cells that improves upon the methods previously used and provides cells isolated in quantity. The present invention provides a list of secreted proteins from omentum mesothelial cells that can be utilized in the described cell based assays.

This application claims priority of U.S. provisional application No. 61/256,770 filed on Nov. 1, 2009 and U.S. provisional application No. 61/259,316 filed on Nov. 9, 2009 included herein in their entirety by reference.

### GOVERNMENT SPONSORED STATEMENT

This invention was made with government support under R44RR024302-02 awarded by National Institutes of Health. The government has certain rights in the invention.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the isolation and use of substantially pure cultures of human omental mesothelial cells. Specifically, the isolation of mesothelial cells is accomplished for utilization, as a screening tool in drug discovery, as a progenitor cell or as a supporting cell for organ regeneration.

2. Description of Related Art

Mesothelial cells have been found to be pivotal in tumor metastasis, peritoneal dialysis, and inflammatory response. These cells are specialized epithelial cells and line both the serous cavities (peritoneal, pericardial, and pleural) and internal organs to provide a frictionless barrier and facilitate the movement of opposing organs and tissues. Ovarian tumor attachments occur through cancer cells binding to peritoneal mesothelial cells and migrating into the surrounding tissue and vasculature. Peritoneal dialysis relies on the intact transport function of mesothelial cells to allow transfer of waste products from the underlying vasculature to the dialysis fluid in the peritoneal cavity. Host response to peritoneal infection is mediated by an inflammatory cascade and cytokines released by peritoneal mesothelial cells. Research into the biology of mesothelial cells continues with the goal of developing methods to provide more effective treatments for disease. Currently, researchers use immortalized cell lines or isolate mesothelial cells from tissue biopsies, pleural effusions, or dialysis effluent.

Mesothelial cells also present a barrier to invading organisms and physical damage. Mesothelial cells are known to provide a frictionless surface for movement of opposing tissues and organs by secreting glycosaminoglycans and surface-active phospholipids, such as phosphatidylcholine. More recent analysis has uncovered a role for mesothelial cells in initiating and resolving inflammatory responses. In a rat model of induced peritonitis, peritoneal mesothelial cells secreted inflammatory cytokines in a temporal manner, rapidly secreting TNFα followed by a secondary release of IL-1β. Following the initial inflammatory response, the rat mesothelial cells secreted the anti-inflammatory cytokine, IL-10, to begin resolution of the response. The greater omentum, the largest peritoneal fold in the abdomen, is directly involved in the initial immune response to peritoneal infection. Mesothelial cells lining the omentum are involved in recruiting neutrophils and macrophages to the peritoneal cavity from the ‘milky spots’ underlying their stomata. This mesothelium also mediates omental attachments that localize infections by sealing off areas of contamination and aid in absorbing microbes and contaminants. Damage to serosal surfaces by infection or surgery can lead to a disruption in the mesothelial layer, fibrin deposition, and peritoneal adhesions. When the mesothelial cell lining is injured, free floating mesothelial cells and those surrounding the site are capable of migrating, implanting, and proliferating to repair the injury. Mesothelial cells migrate as fibroblast-like cells and alter their morphology to the common epithelial ‘cobblestone’ shape upon confluence. Mesothelial cells mediate fibrinolysis through secreting tissue plasminogen activator (tPA), which activates plasminogen to enzymatically degrade fibrin. When the normal repair process is impaired, the deposited fibrin is not removed. This allows fibroblasts to invade and deposit collagen between the opposing surfaces leading to adhesions. According to a 10-year follow up study, surgical adhesions occur in up to 32% of lower-abdominal surgery patients leading to multiple hospital admissions. These relatively large numbers of peritoneal attachments have led researchers to investigate methods to reduce attachment formation by incorporating mesothelial cells or pre-cultured layers of mesothelial cells to site of injury.

Malignant mesothelioma is a rare, but deadly cancer that most commonly occurs in the pleural mesothelium. This site of occurrence for this type of cancer is due mainly because most cases of mesothelioma disease are related to asbestos exposure. Up to 80% of those diagnosed with mesothelioma were exposed to asbestos fibers. This type induction of cancer has a long latency period, with the exposure ranges from 20-50 years prior to diagnosis. This disease caused 2,500 deaths per year in the U.S. between 1999 and 2003, mirroring the U.S. incidence rate of 2,500 cases annually. The high mortality rate is reflective of an average survival time between 6-12 months after diagnosis and no curative treatment being available. In vitro studies using certain types of mesothelial cells have begun to reveal mechanisms leading to mesothelioma, however, more studies are required. Asbestos fibers interact directly with mesothelial cells, where they are ingested and lead to DNA damage from reactive oxygen species. Indirect mechanisms of mesothelial cell damage may also occur due to the generation of inflammatory and angiogenic factors in the pleural cavity. Clearly, there continues to be a need to investigate the mechanisms of action leading to mesothelioma, which could be aided through the availability of a primary mesothelial cell culture system. Epithelial ovarian cancer is highly lethal, due to its advanced stage at primary diagnosis with 70% of women presenting with disease that has spread beyond the ovaries. The primary sites of metastasis are within the peritoneal cavity. Recent investigations have revealed a significant role for mesothelial cells in ovarian cancer progression. Earlier studies showed that ovarian cancer cells could interact with mesothelial cells in vitro forming the basis for implantation and proliferation within the peritoneal cavity. Molecular analysis of the interaction between mesothelial cells and ovarian cancer cells has revealed multiple sets of binding proteins. Mesothelin is a glycoprotein normally expressed by mesothelial cells, but is expressed by a number of cancer cells, including ovarian cancer cells. This protein is being investigated as a target for immunotherapy for ovarian cancer with immunotoxins towards mesothelin showing some promise in culture and xenograft models. Mesothelin has also been implicated as a binding site for ovarian cancer cells mediated through Muc16, a mucin containing the CA125 cancer antigen. An interaction between L1 adhesion molecule on ovarian cancer cells and Neuropilin-1 on mesothelial cells has also been suggested to mediate the implantation of metastatic ovarian cells in the mesothelium. These studies highlight the importance of investigating the role of mesothelial cells in the ovarian cancer progression that may lead to new treatments for the disease.

Of the 450,000 U.S. patients with end-stage renal disease, 6% are undergoing peritoneal dialysis treatment. This treatment relies on the integrity of the peritoneum to facilitate the transfer of metabolic waste products from the bloodstream to dialysis fluid resident in the peritoneal cavity. The patient is able to perform this treatment at home by infusing dialysis fluid into the peritoneal space through a catheter. The fluid remains within the peritoneal cavity from 1 to 4 hours during the day, or 8 to 12 hours overnight before being drained and the process repeated. Damage to the mesothelial cells lining the peritoneal cavity reduces the effectiveness of solute transfer and ultimately results in failure of peritoneal dialysis. Damage to mesothelial cells occurs because they are in direct contact with nonphysiologic dialysis fluid during long dwell times. Osmotic agents, high glucose levels, and low pH contribute to injuring the peritoneum and denuding the surface of mesothelial cells. This increases fibrosis within the mesothelium, disrupting the ultrastructure, and decreasing the effectiveness of peritoneal dialysis. Improvements to peritoneal dialysis fluids have been made to increase their biocompatibility by incorporating neutral pH and lowering glucose degradation products to decrease the oxidative injury to the mesothelium. However, long term peritoneal dialysis continues to result in mesothelial cell injury and ultimate failure of the treatment. Exposure of the mesothelial cell layer to the components of the dialysis fluid also induces an epithelial to mesenchymal transition (EMT) in these cells. This process degrades the tight junctions between cells, causes a morphology change to fibroblast-like cells, and increases their migratory and invasive properties. Overall, EMT decreases the integrity of the peritoneal lining greatly increasing transport rates and rendering peritoneal dialysis ineffective. The mechanism of EMT is still under investigation, however, it appears to involve glucose degradation products and TGF-β related signaling pathways. Further, analysis of mesothelial cell EMT has the potential to identify methods of blocking transdifferentiation and extending the utility of peritoneal dialysis.

In U.S. Pat. No. 6,987,024, there is described the isolation and use for pharmacological research of ovarian mesothelial cells. They are described as useful in the characterization of ovarian cancer and to generate a human ovarian tissue model. No detailed isolation of omentum-derived mesothelial cells is disclosed or suggested.

Omentum mesothelial cells have been suggested to play a role in diabetes and obesity, and appear to have some function in the inflammatory process related to these disorders. Further analysis of this response could lead to identification of methods leading to treatment of obesity and/or diabetes. Accordingly, there exists a need for methods to identify, isolate, culture, characterize, and utilize in research, omentum, and other organ derived mesothelial cells that retain their functional capabilities.

### BRIEF SUMMARY OF THE INVENTION

The invention relates to the production, characterization, and isolation of a population of substantially pure human omentum mesothelial cells that retain the desired characteristics of the original in vivo cells activity and capability, such as to secrete proteins, which can be used as disease indicators, react to stimulus from test compositions, co-culture with other cells, such as adipose cells and can be used as a screening tool in drug discovery. It also relates to the production of methods relating to use as a tissue model for cells and for methods of providing cell therapy by introduction into a recipient in a location the cells can act for support in growing organs, i.e., organ regeneration. This also applies to use of the cells as a supporting cell in organ regeneration, either in vivo or ex vivo. This also applies to the use of the cells as a supporting cell in soft tissue repair and/or soft tissue generation in vivo or ex vivo.

Accordingly, in one embodiment of the present invention, there is disclosed a method of providing a source of omentum mesothelial cells comprising providing a substantially pure isolated population of human omentum mesothelial cells, and using the cells or any part of the cells thereof as a target for a selected drug under development.

In yet another embodiment, there is disclosed a method of isolating mesothelial cells comprising:
a) isolating tissue containing mesothelial cells;
b) treating the tissue with an enzyme solution that separates mesothelial cells from other cell types;
c) removing the tissue from the solution; and
d) centrifuging the solution to isolate the mesothelial cells.

Another embodiment of the present invention includes a method of providing a source of omentum, myocardial, liver, lung, renal, peritoneal, intestinal or pancreatic or mesothelial cells comprising providing a substantially pure isolated population of human omentum, myocardial, liver, lung, renal, peritoneal, intestinal or pancreatic mesothelial cells and using the cells in a selected bioassay.

Another embodiment of the present invention includes a method of providing a source of omentum, myocardial, liver, lung, renal, peritoneal, intestinal or pancreatic mesothelial cells comprising providing a substantially pure isolated population of human omentum, myocardial, liver, lung, renal, peritoneal, intestinal or pancreatic mesothelial cells and using at least a portion of the cells in a selected cell therapy.

Another embodiment of the present invention comprises a method of providing a source of omentum, myocardial, liver, lung, renal, peritoneal, intestinal or pancreatic mesothelial cells comprising providing a substantially pure isolated population of human omentum, myocardial, liver, lung, renal, peritoneal, intestinal or pancreatic mesothelial cells and using the cells to co-culture with another selected cell and then observing the effect on the selected cell.

Another embodiment of the present invention comprises a method of providing a source of omentum mesothelial cells comprising providing a substantially pure isolated population of human omentum mesothelial cells, and isolation of the conditioned medium from the substantially pure population of omentum mesothelial cells, and using the conditioned medium to treat another selected cell, and then observing the effect on the selected cell.

Another embodiment of the present invention includes a method of providing a source of proteins for a bioassay comprising isolating proteins from an isolated population of human omentum, myocardial, liver, lung, renal, peritoneal, intestinal or pancreatic mesothelial cells and using the proteins as at least one component in the bioassay.

Another embodiment of the present invention includes a method of determining the effect of mesothelial cell proteins on adipocytes comprising co-culturing isolated human omentum, myocardial, liver, lung, renal, peritoneal, intestinal or pancreatic mesothelial cells with human adipocytes.

Another embodiment of the present invention includes a method of growing soft tissue or organ tissue of a human comprising using human omental mesothelial cells as support cells while growing the human tissue in vitro or ex vivo.

### BRIEF DESCRIPTION OF THE DRAWINGS

FIGS. 1a and 1b are a depiction of the characterization of mesothelial cells.

FIGS. 2a and 2b are a further characterization of mesothelial cells.

FIG. 3 is a depiction of mesothelial cells expressing omentin and visfatin.

FIG. 4 is the result of the secretion of visfatin in the presence of other composition over time.

FIG. 5 shows that mesothelial cells are not enriched for CD31.

FIG. 6 shows the results of co-culturing mesothelial cells with subcutaneous adipocytes.

FIG. 7 shows the results of co-culturing mesothelial cells with omental adipocytes.

FIG. 8 and FIG. 9 are profiles of a limited set of secreted proteins from mesothelial cells and omental adipocytes.

FIG. 10 and FIG. 11 are profiles of an additional limited set of secreted proteins from mesothelial cells.

FIG. 12 shows neuropeptide Y (NPY) secretion from omental mesothelial cells.

FIG. 13 shows detection of secreted molecules present in conditioned media isolated from omental mesothelial cells using an antibody array.

FIG. 14 shows the induction of NR5A2 expression by TGF-beta in human omentum mesothelial cells during epithelial to mesenchymal transition.

### DETAILED DESCRIPTION OF THE INVENTION

While this invention is susceptible to embodiment in many different forms, there is shown in the drawings and will herein, be described in detail specific embodiments, with the understanding that the present disclosure of such embodiments is to be considered as an example of the principles, and not intended to limit the invention to the specific embodiments shown and described. In the description below, like reference numerals are used to describe the same, similar, or corresponding parts in the several views of the drawings. This detailed description defines the meaning of the terms used herein, and specifically describes embodiments, in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases, or in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps, or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function, and that one skilled in the art could select from these or their equivalent in view of the disclosure herein, and use of the term “means” is not intended to be limiting.

The practice of the present invention will employ, unless otherwise indicated, conventional, techniques of immunology, molecular biology, cell biology, and recombinant DNA technology, which are within the skill in the art. (See U.S. Pat. No. 6,987,024 incorporated herein, in its entirety by reference.)

As used herein, “human omentum mesothelial cells” or “omental mesothelial cells” refers to primary human cells derived from the greater and/or lesser omentum of the peritoneum in the abdomen. These are terminally differentiated or pre-determined omental mesothelial cell types (subconfluent and confluent), which have become or will become differentiated as omental mesothelial cells, and no longer reside as having a pluripotent or multipotent capacity cell, unless otherwise modified to do so; for example, derivation of a pluripotent cell from a differentiated mesothelial cell by chemical or genetic modification (i.e., induced pluripotent stem cells). Also, mesothelial cells can undergo induced transdifferentiation, such as the epithelial-to-mesenchymal transition. Mesothelial cells are involved in recruiting neutrophils and macrophages to the peritoneal cavity from the milky spots underlying their stomata. These cells also mediate omental attachments that localize infections by sealing off areas of contamination, and aid in absorbing microbes and contaminants. As described herein, mesothelial cells also have the potential of regulating growth and function of surrounding cells and associated organ tissues.

As used herein, additional sources of human mesothelial cells include: “human myocardial mesothelial cells” refer to primary human cells derived from myocardial tissue, including the pericardium; “human renal mesothelial cells” refer to primary human cells derived from the kidney; “human peritoneal mesothelial cells” refer to primary human cells derived from regions of the peritoneum other than the omentum; “human intestinal mesothelial cells” refer to primary human cells derived from the outer layer of cells from the intestines and stomach; “human liver mesothelial cells” refer to primary human cells derived from liver tissue; “human lung mesothelial cells” refer to primary human cells derived from the pleural cavity; and “human pancreatic mesothelial cells” refer to primary human cells derived from the pancreas. These cells are differentiated or pre-determined mesothelial cell types (subconfluent and confluent), which have become or will become differentiated as mesothelial cells and no longer reside as having a pluripotent or multipotent capacity cell, unless otherwise modified to do so; for example, derivation of a pluripotent cell from a differentiated mesothelial cell by chemical or genetic modification (i.e., induced pluripotent stem cells).

The term “heterologous”, as applied to a cell used for immunization or transplantation means that the cell is derived from a genotypically distinct entity from the recipient. For example, a heterologous cell may be derived from a different species or a different individual from the same species as the recipient. An embryonic cell derived from an individual of one species is heterologous to an adult of the same species. “Heterologous”, as applied to a recipient means that the recipient is a genotypically distinct entity from the source of the cells that are being introduced into the recipient.

A cell is of “ectodermal”, “endodermal”, or “mesodermal” origin, if the cell is derived, respectively, from one of the three germ layers, the ectoderm, the endoderm, or the mesoderm of an embryo. The ectoderm is the outer layer that produces the cells of the epidermis, and the nervous system. The endoderm is the inner layer that produces the lining of the digestive tube, and its associated organs. The middle layer, mesoderm, gives rise to several organs, including, but not limited to, heart, kidney, mesothelium, and gonads, connective tissues (e.g., bone, muscles, tendons), and the blood cells.

As used herein, a “substantially pure” isolated population of mesothelial cells is a population of cells that is comprised at least about 85% omental mesothelial cells, preferably at least about 90%, and even more preferably at least about 95% or more.

The terms “medium”, “cell culture medium”, and “culture medium” are used interchangeably. The terms refer to the aqueous microenvironment, in which the mammalian cells are grown in culture. The medium comprises the physicochemical, nutritional, and hormonal microenvironment.

A “defined medium,” “basal cell-sustaining medium,” “nutrient medium”, and “basal nutrient medium” are used interchangeably herein, and refer to a medium comprising nutritional and hormonal requirements necessary for the survival and/or growth of the cells in culture, such that the components of the medium are known. Traditionally, the defined medium has been formulated by the addition of nutritional and growth factors necessary for growth and/or survival. Typically, the defined medium provides at least one component from one or more of the following categories: a) all essential amino acids, and usually the basic set of twenty amino acids plus cystine; b) an energy source, usually in the form of a carbohydrate, such as glucose; c) vitamins and/or other organic compounds required at low concentrations; d) free fatty acids; and e) trace elements, where trace elements are defined as inorganic compounds or naturally occurring elements that are typically required at very low concentrations, usually in the micromolar range. The defined medium may also optionally be supplemented with one or more components from any of the following categories: a) one or more mitogenic agents; b) salts and buffers as, for example, calcium, magnesium, and phosphate; c) nucleosides and bases such as, for example, adenosine and thymidine, hypoxanthine; and d) protein and tissue hydrolysates including extracellular matrix components such as collagen and laminin.

As used herein, “conditioned media” refers to culture media, free of intact cells, in which mesothelial cells have been grown or been contacted. Mesothelial cells grown in nutrient media may release factors which promote the continued survival, growth, and maintenance of pre-existing state of pre-differentiation of the mesothelial cells and surrounding cells and tissues. Conditioned media may be used to reconstitute a cell pellet or added to cells already existing in culture plates. Conditioned media may also be used alone, or to supplement nutrient media being used to feed mesothelial cells.

“Standard incubation conditions” as used herein” refers to the physicochemical conditions in an incubator designed for tissue culture, in which cells are placed. Generally, the standard incubation conditions are about 37 degrees Celsius and about 5% CO₂ content with humidification. All tissue culture techniques and equipment should be performed under sterile conditions.

“Serum”, as used herein, refers to the fluid phase of mammalian blood that remains after blood is allowed to clot.

“Serum biomolecules”, as used herein, refers to biological compositions found in serum. Examples include, but are not limited to, albumin, alpha1-globulin, alpha 2-globulin, beta-globulin, gamma-globulin, insulin-like growth factor 1, insulin, transforming growth factors, fibroblast growth factors, and epidermal growth factor. Serum biomolecules can include biological compositions, whole or partial, that are either naturally found in serum, or derived from processing and handling of serum. Proteins isolated from mesothelial cells or from conditioned media from mesothelial cells include, but are not limited to, Activin C (INHBC), AXL receptor tyrosine kinase (AXL), Cancer Antigen 125 (MUC16), Carbohydrate Antigen 19-9, chemokine C-C motif ligand 28 (CCL28), CD30 Ligand (TNFSF8), cysteine rich transmembrane bone morphogenic regulator 1 (CRIM1), c-src tyrosine kinase (CSK), Decorin (DCN), dickkopf homolog 1 (Dkk1), Ectodysplasin A (EDA), epidermal growth factor receptor (EGFR), aminoacyl tRNA synthetase complex-interacting multifunctional protein 1 (AIMP1), chemokine C-X-C motif ligand 5 (CXCL5), Endostatin (COL18A1), Endothelin (EDN1), PANDER (FAM3B), fibroblast growth factor 2 (FGF2), fibroblast growth factor 11 (FGF11), fibroblast growth factor 16 (FGF16), fibroblast growth factor 7 (FGF7), Follistatin (FST), Follistatin-like 1 (FSTL1), Galectin-3 (LGALS3), colony stimulating factor 3 (CSF3), growth differentiation factor 3 (GDF3), growth differentiation factor 5 (GDF5), growth differentiation factor 9 (GDF9), Glypican 3 (GPC3), Glypican 5 (GPC5), GREMLIN (GREM1), chemokine C-X-C motif ligand 1 (CXCL1), intercellular adhesion molecule 1 (ICAM1), insulin-like growth factor binding protein 2 (IGFBP2), insulin-like growth factor binding protein 3 (IGFBP3), insulin-like growth factor binding protein 6 (IGFBP-6), insulin-like growth factor binding protein 7 (IGFBP7), insulin-like growth factor 2 receptor (IGF2 R), interleukin 1 alpha (IL1A), interleukin 1 family member 6 (IL1F6), interleukin 1 family member 9 (IL1F9), interleukin 15 receptor alpha (IL15RA), interleukin 17 (IL17A), interleukin 25 (IL25), interleukin 23 (IL23), interleukin 3 (IL3), interleukin 4 (IL4), interleukin 6 (IL6), interleukin 7 (IL7), interleukin 8 (IL8), interleukin 9 (IL9), kringle containing transmembrane protein 2 (Kremen-2), lipoprotein (LPA), low density lipoprotein receptor-related protein 1 (LRP1), low density lipoprotein receptor-related protein 6 (LRP6), chemokine C-C motif ligand 2 (CCL2), colony stimulating factor 1 (CSF1), chemokine C-X-C motif ligand 2 (CXCL2), matrix metallopeptidase 1 (MMP1), matrix metallopeptidase 10 (MMP10), matrix metallopeptidase 11 (MMP11), neuregulin 3 (NRG3), oncostatin M (OSM), tumor necrosis factor receptor superfamily member 11b (TNFRSF11B), pregnancy-associated plasma protein A (PAPPA), pentraxin 3 (PTX3), granulin (GRN), chemokine C-X-C motif ligand 12 (CXCL12), secreted frizzled-related protein 4 (SFRP4), interleukin 6 signal transducer and soluble interleukin 6 signal transducer (IL6ST), synaptotagmin-like 1 (STYLI), SMAD family member 4 (SMAD4), secreted protein acidic cysteine-rich (SPARC), tissue factor pathway inhibitor (TFPI), Thrombospondin\thrombospondin 1 (THBS1), TIMP metallopeptidase inhibitor 1 (TIMP1), TIMP metallopeptidase inhibitor 2 (TIMP2), tumor necrosis factor receptor superfamily member 1A (TNFRSF1A), vasorin (VASN), vascular cell adhesion molecule 1 (VCAM1), vascular endothelial growth factor A (VEGFA), vascular endothelial growth factor C (VEGFC), ectodysplasin A2 receptor (EDA2R), visfatin (NAMPT), omentin (ITLN1). Depending on whether the mesothelial cells are from the omentum or additional sources, they will have various proteins and in differing amounts.

As used herein, “diseases associated with use of omental, myocardial, liver, lung, renal, peritoneal, intestinal or pancreatic mesothelial cells” include metabolic diseases, such as obesity, cardiovascular disease, atherosclerosis, type 2 diabetes, type 1 diabetes and fatty liver disease.

As used herein, “co-culture” includes two or more cell types directly or indirectly contacted with each other such that a) factors from either or each are contacted with the other by sharing the same medium or being contacted by conditioned medium, b) direct cell-cell contact allowing physical, mechanical, chemical, or biochemical interactions to influence cellular characteristics. “Cells for co-culture with mesothelial cells” of the present invention include cells, which when co-cultured are useful for investigation of the effects of the plurality of the mesothelial secreted proteins on cell growth and function. For example, co-culturing omental mesothelial cells with adipocytes results in the adipocytes accumulating more lipid, such as triglycerides. Other examples of co-culture with mesothelial cells include, but are not limited to, cells or tissue isolated from: a) liver, for the study of hepatic growth and function related to hepatotixicity, fatty liver disease, and glucose metabolism; b) pancreas, for the study of beta-cell growth and maintenance and glucose mediated insulin secretion; c) cardiac, for the study of cardiomyocyte growth and cardiac function; and d) pleural cells, for the study of lung function and associated diseases.

Isolation and Maintenance of Primary Human Omental Mesothelial Cells

Omental mesothelial cells of the present invention are isolated from tissue from the greater and/or lesser omentum. Omental cells can be isolated from diabetic individuals, lean individuals, or the like. The tissue can be identified initially by gross anatomy, outward appearance, location, or other means as desired. Once the appropriate tissue is identified, the tissue is washed with a nutrient medium and then microdissected into appropriate size. This can be done with appropriate devices, such as scalpels, forceps, and the like, as well as ultrasonic devices. Next, the tissue sample is treated with an enzyme-containing solution that isolates the mesothelial cells from the preadipocytes. An example of an appropriate enzyme is trypsin, in one embodiment a solution of 0.25% trypsin, which can be added to the tissue in roughly equal volumes. The tissue is kept in contact with the enzyme an appropriate amount of time to complete the separation; for example, in the case of 0.25% trypsin contact should be for about 20 minutes at 23-25° C. Cells can be isolated from the resulting mixture by centrifuging the enzyme cell mixture at an appropriate speed, time, and temperature. One skilled in the art will be able to effectively maximize each of these criteria for centrifugation. In one embodiment, the mixture is spun at 1200 rpm for five minutes at 20 degrees C. The resulting pellet containing mesothelial cells is resuspended in DMEM-F12 medium containing fetal bovine serum (5-10%) and in one embodiment may contain epidermal growth factor (5 ng/ml) and may contain in another embodiment platelet-derived growth factor-BB (10 ng/ml). The resuspended cells are plated in tissue cultureware. Mesothelial cells are then expanded in Media 199 containing 5% fetal bovine serum and 10 ng/ml platelet-derived growth factor-BB.

Mesothelial cells of this invention are maintained after isolation in basal nutrient media; Media 199, containing 5% fetal bovine serum which may contain 10 ng/ml platelet-derived growth factor-BB. Different types of substrate on tissue culture plates can be used. Mesothelial cells of this invention may be cultured in serum-free nutrient media or serum-containing nutrient media. As is well-known to those of ordinary skill in the art, serum is commonly added to nutrient media to further enhance cell growth. Serum contains many serum biomolecules; however, the mesothelial cells of this invention may be grown in the absence of a plurality of these serum biomolecules. Cell growth of mesothelial cells may be enhanced by the addition of one or more proteins found in serum, for example, but not limited to, bovine serum albumin (or BSA), platelet-derived growth factor, epidermal growth factor, fibroblast growth factor, insulin-like growth factor 1, insulin, hepatocyte growth factor, and transforming growth factors.

The frequency of feeding mesothelial cells may be once a day or every other day. In one embodiment, mesothelial cells may be fed by replacing the entirety of the old nutrient media with new nutrient media. In another embodiment, mesothelial cells may be fed with conditioned media in which these cells were grown. Because the claimed mesothelial cells are unique to this invention, and will secrete factors specific to these cells, the conditioned media derived from the mesothelial cells are also unique. In one embodiment of the invention, cell to cell contact of mesothelial cells to each other is maintained throughout the culturing of mesothelial cells to promote a higher proliferation rate. Addition of conditioned media may also promote better growth of the mesothelial cells. A skilled artisan can determine if the addition of conditioned media is advantageous to the growth of the specific mesothelial cells by supplementing the nutrient media stepwise with an increasing amount of conditioned media. Cell growth can be determined by counting the number of cells per volume of media before and after the addition of conditioned media. Alternatively, cell viability (e.g., trypan blue) can be used to assess, if addition of conditioned media to the culturing condition is advantageous to the growth of the mesothelial cells. A frequency of feeding, that is preferable for promoting the survival and growth of mesothelial cells is once a week, even more preferably is twice a week, and most preferably is every other day. Mesothelial cells can be kept in culture for at least four passages. Passaging cultured cells is a common procedure known to those skilled in the art. An example includes washing the cells with phosphate buffered saline, incubating the cells with a solution containing 0.25% trypsin to detach the cells from the cultureware and reseeding the suspended cells at varying densities in basal nutrient media. The mesothelial cells can also be stored indefinitely while frozen in liquid nitrogen. An example of the storage medium is Media 199 containing 20% fetal bovine serum and 7.5% dimethyl sulfoxide (DMSO).

### Characterization of Omental Mesothelial Cells

The population of mesothelial cells of this invention are isolated as described above and have several defining characteristics. The identification of cells of the present invention especially involves the isolation of cells that have already begun or have differentiated.

The identification of the mesothelial cells may be accomplished by morphology or specific markers or combinations of these types of techniques. Markers that can be used to detect omental mesothelial cells and distinguish them from potential contaminating cells include, but are not limited to, mesothelin, omentin, NPY, podoplanin, CD31 negative, and CD45 negative. Markers to detect the mesothelial cells can be used in direct and indirect immunofluorescence, immunohistochemistry, immunoblotting and flow cytometry among others. In one embodiment of the present invention highly enriched levels of omentin, mesothelin and NPY in omental mesothelial cells are indicative of characterization.

### Uses of the Mesothelial Cells of the Present Invention

### Use of Cells for Drug Development and Screening

The cells described in the present invention could be used in a drug development and screening programs including gene expression studies. Because it is known that cells can change their functional status (e.g. secrete or produce more or less of a particular protein or set of proteins) in disease states, the cells can be useful in screening and developing drugs for those diseases. Thus, since omentum mesothelial cells are involved in diabetes and obesity, they can be used to screen for changes in protein expression as a means of detecting drugs useful for treatment of these diseases.

By starting with mesothelial cells of the invention with known characteristics, the cells, portion of the cells, proteins secreted or isolated from the cells, or the like can be treated with a candidate drug, and the cells or result observed to determine if there is a beneficial change. For example, test candidates can be administered to mesothelial cells isolated from an individual with type 2 diabetes and the proteins produced and measured before and after treatment then compared to find candidates for treating the disease state. Numerous proteins from the mesothelial cells could be examined (e.g. those listed in claim 13). Additional examples of monitoring cells could include, but are not limited to, monitoring cell growth, cell death, and activation or inhibition of a cellular process and the like.

The cells could be used to test for candidates to prevent tumor cells from binding during metastasis to the peritoneal cavity. In addition, the cells could be studied for the ability to secrete factors that initialize and\or resolve inflammatory responses. These cells are involved in fluid removal from the peritoneal space and are critical during dialysis. The mechanism for aiding or supporting these actions can be studied in drug candidates with these cells.

### Uses of Mesothelial Cells in Bioassays

The mesothelial cells disclosed in the present invention can be used in a wide variety of bioassays. In one example, the cells could be screened for endogenously expressed secreted proteins, such as cytokines and the presence of and/or the quantification of cells as an indicator for disease, such as obesity, type 2 diabetes, and cardiovascular disease. The cells could be used as a screening platform for cell surface proteins, such as cell surface receptors, for example, adiponectin receptors. In other embodiments, the cells could be used for specifically screening for the presence of specific compositions, such as proteins, for example, those listed in claim 13.

Protein-protein interactions can be determined with techniques, such as yeast two-hybrid systems or immunoprecipitation followed by immunoblots. Proteins from the cells can be used to identify other unknown proteins, or other cell types that interact with the mesothelial cells of the present invention. Examples of these unknown proteins include, but are not limited to, growth factors, hormones, enzymes, transcription factors, translational factors, kinases, phosphatases, nuclear hormone receptors, cell-surface proteins, and tumor suppressors.

### Uses of Mesothelial Cells for Cell Therapy

The mesothelial cells of the present invention can be used in various cell type therapies. For example, they could be used to treat injury to the parenteral walls, where injury has occurred. In other cell therapy applications, one could use the cells in organ regeneration and/or soft tissue repair. These cells giving sufficient number could be used as a supporting cell for growing internal organs, such as liver, pancreas, cardiac, and lung ex vivo. For example, organ liver regeneration can be performed in a laboratory environment using a bioartificial liver device. In one embodiment, the omental mesothelial cells could be incorporated into such a device to aid in the support of liver regeneration by providing the necessary extracellular microenvironment, and secreted soluble factors that enhance liver growth and development.

Mesothelial cells can undergo epithelial to mesenchymal transition. NR5A2 is induced in this process and provides an example of the ability of these cells to become more pluripotent.

Co-Culturing with Other Cell-Types

The omental mesothelial cells of the present invention can be co-cultured with other cells to see if the mesothelial cells, or the test cells have any positive or negative effect on the other cell in the co-culture. Cell types derived from organs and tissues, such as liver, pancreas, adipose, cardiac, and plural cells could be utilized in a co-culturing assay. In this method, the cells are allowed to grow together in direct contact or separated by a Transwell filter for a period of time. The effect of one cell on the other is measured. Thus, for example, the production of proteins positively or negatively, the particular activity of the cells, growth rates, survival rates, and the like could all be measured before and after co-culturing. Also, cell-type specific functions could be modulated in the co-culture system. For example, when co-cultured with omental mesothelial cells, adipocytes accumulate more lipid. Other examples would include, but not limited to, enhancement of liver or hepatocyte function, and enhancement of pancreas or pancreas-derived insulin-secreting Beta cell growth or function, when co-cultured with omental mesothelial cells.

### EXAMPLES

### Isolation and Culturing Omental Mesothelial Cells

### Example 1

A sample approximately 10 grams of omental tissue is removed from the greater or lesser omentum of a subject. The tissue piece is transferred to a cryovial and placed in a liquid nitrogen freezer for long term storage or used immediately for mesothelial cell isolation.

The thawed or freshly isolated tissue sample is tested for pathogens and the test sample precisely weighed. The tissue sample is then mixed with an equal volume of a solution containing of 0.25% trypsin and incubated undisturbed for 20 minutes at 23-25° C. The tissue fragments are then removed from the solution and the trypsin solution containing the mesothelial cell suspension is centrifuged at 1200 rpm for 5 minutes at 20° C. The resulting pellet containing mesothelial cells is resuspended in DMEM-F12 medium containing fetal bovine serum (5-10%) and epidermal growth factor (5 ng/ml), and cells are plated in tissue cultureware. Mesothelial cells can then be expanded in Media 199 containing 5% fetal bovine serum and growth factors such as 10 ng/nl PDGF-BB or 5 ng/ml epidermal growth factor).

### Example 2

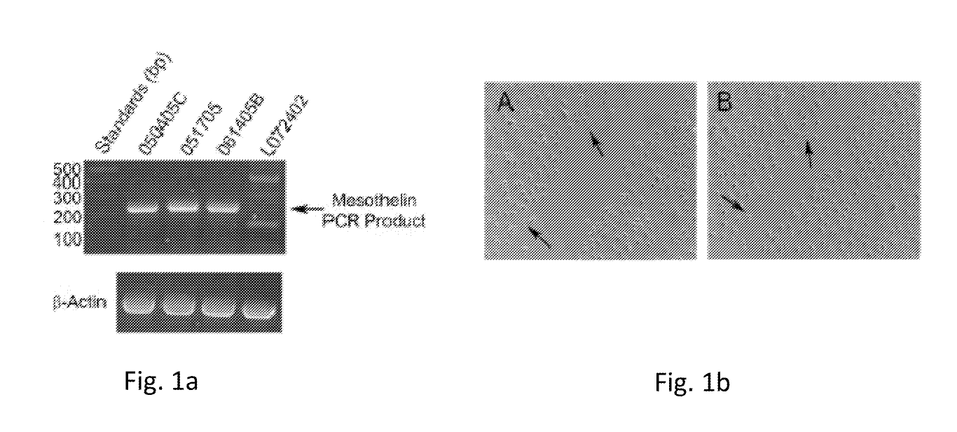

Mesothelin is expressed in the purified omental mesothelial cells. This was shown by reverse-transcription polymerase chain reaction (RT-PCR) assay, using RNA isolated from 3 unique donor lots of omental mesothelial cells. FIG. 1a shows mesothelin expression analyzed by RT-PCR. 1 microgram of total RNA from three mesothelial lots and one preadipocytes lot (L072402) was used as a template for RT-PCR reaction. Primers for mesothelin or Beta-actin were added to assess expression of each gene.

### Example 3

Mesothelial cells are known to be involved in wound healing. As shown in FIG. 1b, mesothelial cells were grown on collagen coated plates and the cell monolayer scrapped to mimic wounding. Fibroblastic mesothelial cells were recruited to the wound site after 3 hours (A) and 18 hours (B). Fibroblast mesothelial cells are indicated by arrows.

### Example 4

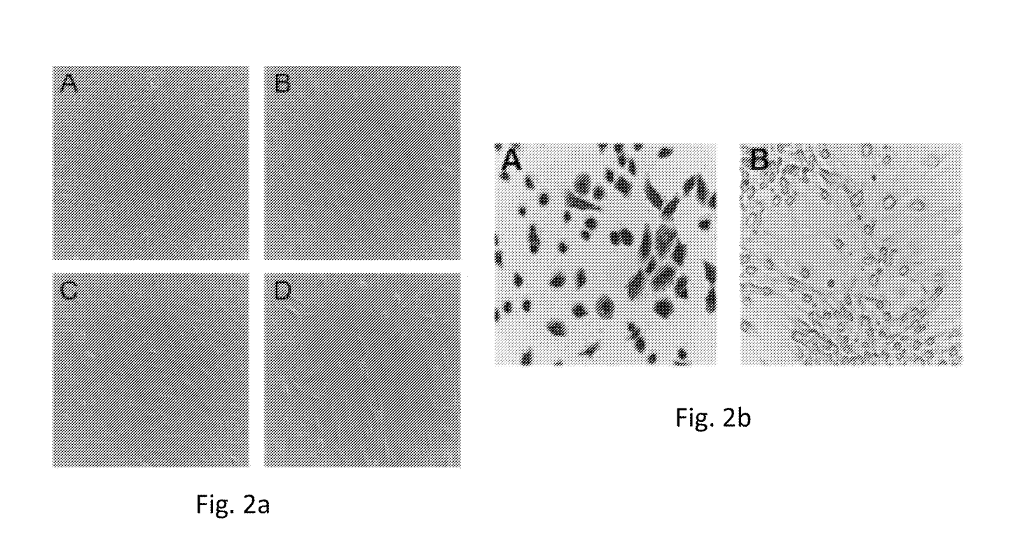

FIG. 2a shows the characteristic transforming growth factor beta (TGF-β) induced morphological change that occurs during the epithelial to mesenchymal transition typical of mesothelial cells. Mesothelial cells were grown in either Growth Medium (A) or TGF-beta (B,C, and D) Medium for passages 2 (A,B), 3(C), and 4 (F). TGF-beta treated cells acquire a fibroblast phenotype.

### Example 5

Mesothelial cells are known to express specific cytokeratins. In FIG. 2b, mesothelial cells were subjected to immunohistochemical staining, using a mixture of antibodies specific for cytokeratins 7, 8, 18, and 19. Positive staining was visualized, using a secondary antibody conjugated to alkaline phosphatase.

### Example 6

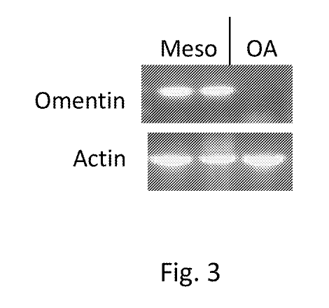

In FIG. 3, we show that omental mesothelial cells express the mRNA coding for omentin. This was accomplished by RT-PCR, using RNA isolated from mesothelial cells. In contrast, we did not detect omentin mRNA in omental adipocytes.

### Example 7

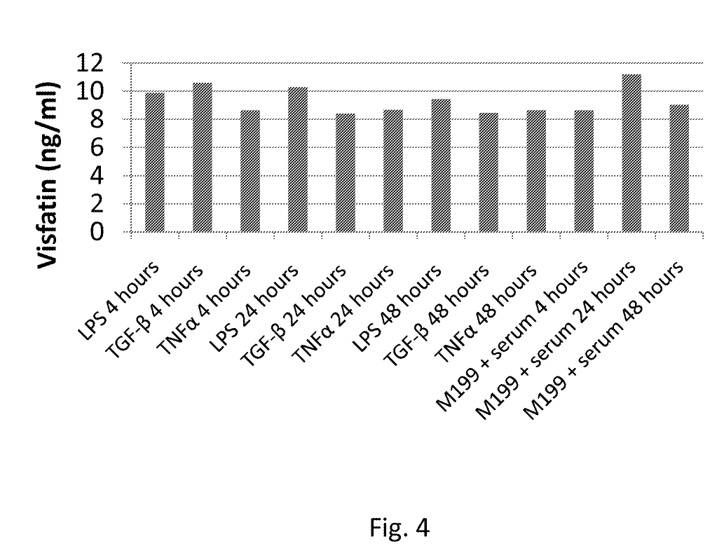

Monolayer cultures of omental mesothelial cells were incubated in Media 199, containing fetal bovine serum, lipopolysaccharide, TGF-beta, or TNF-alpha for 4, 24, or 48 hours. The cell-free conditioned media was subjected to analysis for secreted visfatin, using an enzyme-linked immunosorbant assay (ELISA) specific for human visfatin. The data indicate that visfatin is produced and secreted from mesothelial cells. See FIG. 4.

### Example 8

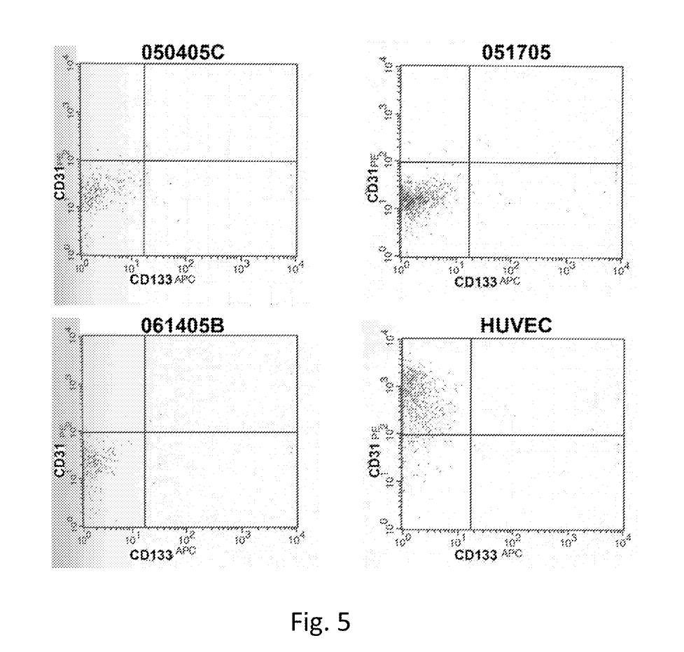

Three unique donor lots of mesothelial cells (050405C, 051705, and 061405B) and human umbilical vein endothelial cells (HUVEC) were analyzed for the presence of the endothelial marker, CD31. The cells were incubated with an antibody specific for CD31 coupled to a fluorescent reporter and analyzed by flow cytometry. These data shown in FIG. 5 indicate that mesothelial cells do not express markers indicative of endothelial cells, whereas the HUVEC cells are positive for CD31 as expected.

### Examples 9

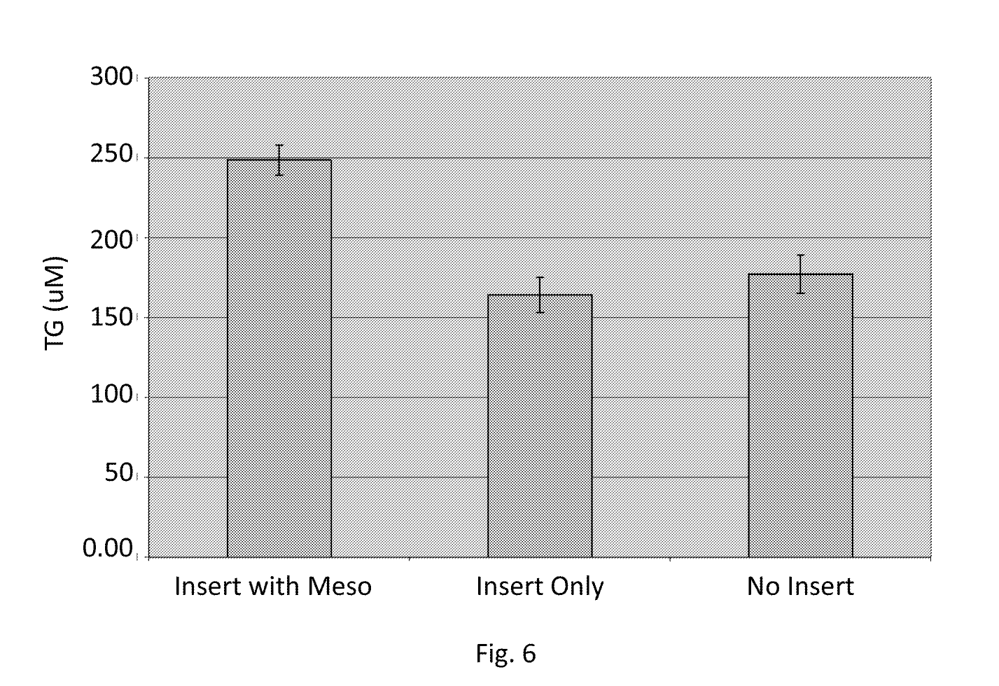

Human subcutaneous pre-adipocytes were induced to undergo adipocyte differentiation in the absence or presence of co-cultured omental mesothelial cells. These experiments were performed, using mesothelial cells grown on transwell inserts in the presence of the pre-adipocytes for 14 days. The inserts allow for exchange of secreted factors between the cell-types, but there is no direct cell-cell contact. At the end of the incubation, triglyceride content was determined in the adipocytes. The data indicates that secreted factors present only in the co-cultures mediate a 50% increase in accumulated lipid in the adipocytes (FIG. 6).

### Example 10

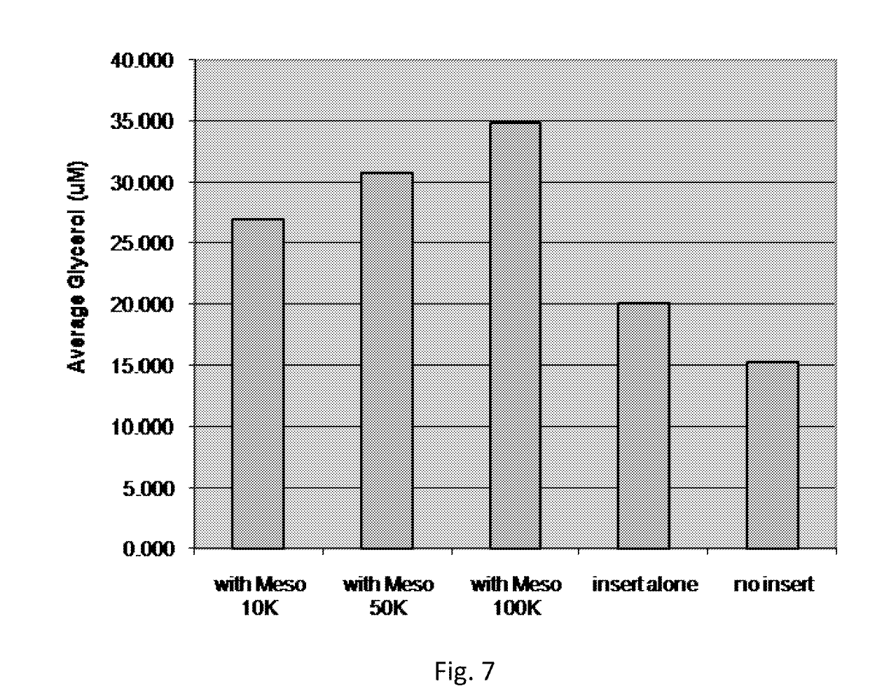

Human omental pre-adipocytes were induced to undergo adipocyte differentiation in the absence or presence of co-cultured omental mesothelial cells at various concentrations of mesothelial cells (10,000, 50,000, and or 100,000 mesothelial cells/well). These experiments were performed, using mesothelial cells grown on transwell inserts in the presence of the pre-adipocytes for 14 days. The inserts allow for exchange of secreted factors between the cell-types, but there is no direct cell-cell contact. At the end of the incubation, triglyceride content was determined in the adipocytes (FIG. 7). The data indicates that secreted factors present only in the co-cultures mediate a dose dependent increase (up to 75%) in accumulated lipid in the adipocytes.

### Example 11

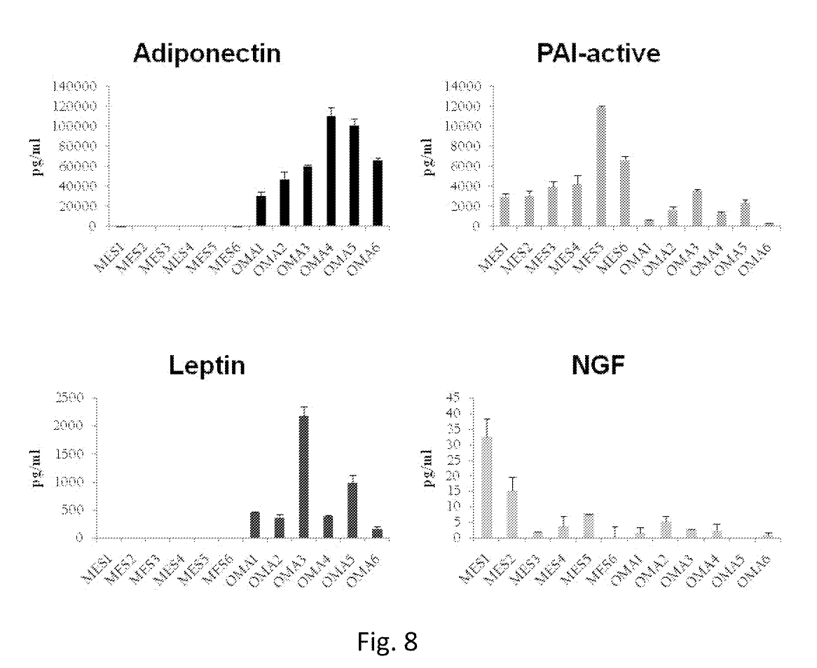

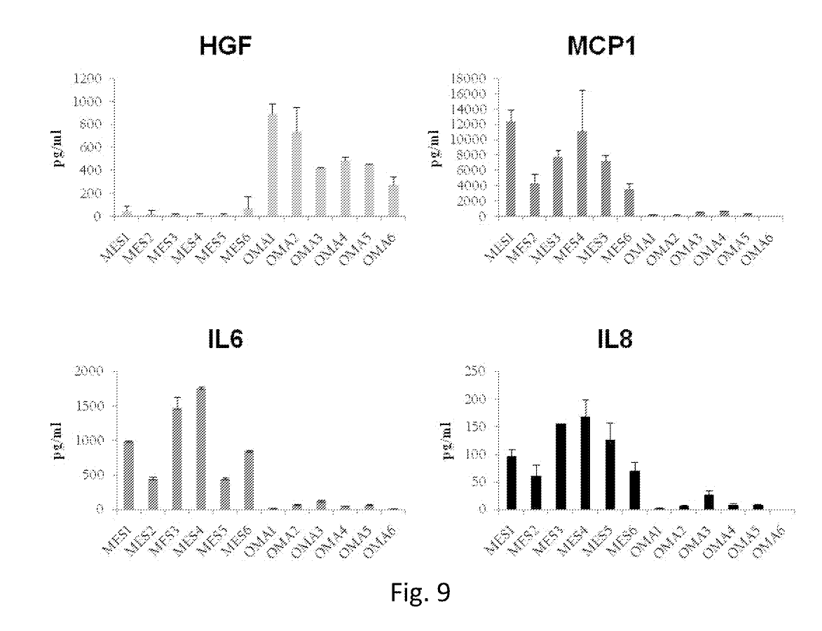

Conditioned media was collected after an overnight incubation in serum free media and analyzed, using a Luminex analytical platform specific for the human forms of the indicated secreted proteins. Six unique donor lots of purified omental mesothelial cells (MES1-6) and six unique lots of omental adipocytes (OMA1-6) were used for these experiments as shown in FIG. 8 and FIG. 9.

### Example 12

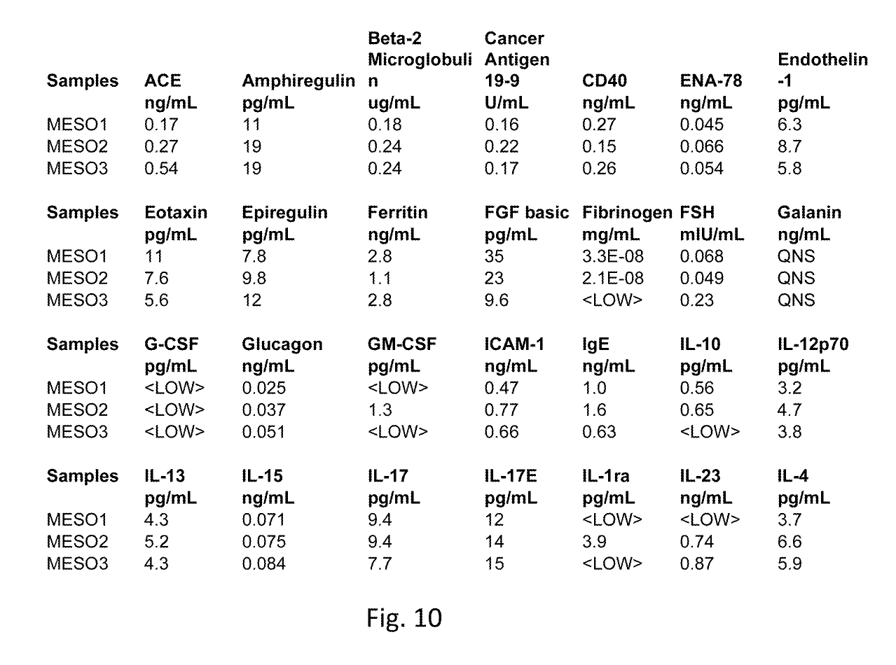

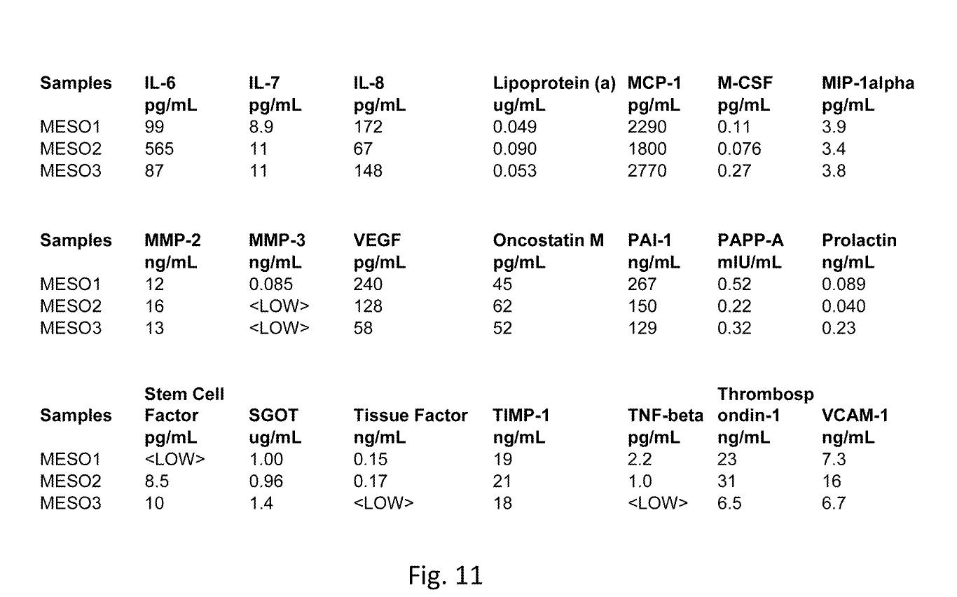

Conditioned media was collected after an overnight incubation in serum free media and analyzed, using a Luminex analytical platform specific for the human forms of the indicated secreted proteins. Nine unique donor lots of purified omental mesothelial cells (MESO1-9) were used for these experiments as shown in FIG. 10 and FIG. 11.

### Example 13

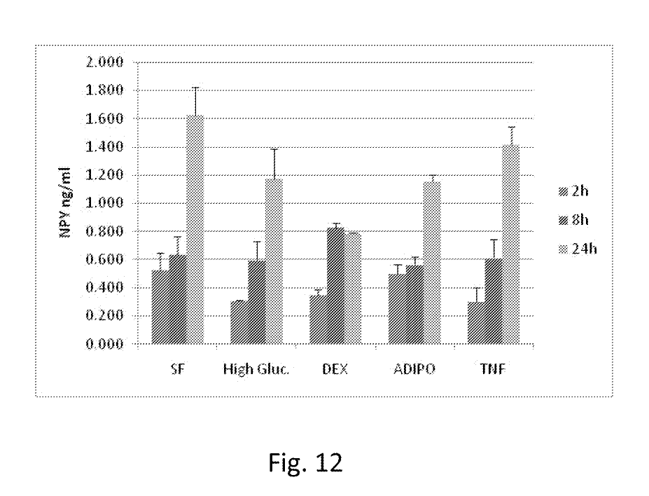

Mesothelial cells were incubated for 2, 8, or 24 hours in serum free media (SF), or in SF containing: a) 25 mM Glucose (High Gluc.); b) 5 uM dexamethasone (DEX); c) 2.5 ug/ml adiponectin (ADIPO); and d) 10 ng/ml tumor necrosis factor alpha (TNF). Levels of secreted NPY in the conditioned media were determined using a human NPY specific ELISA (FIG. 12).

### Example 14

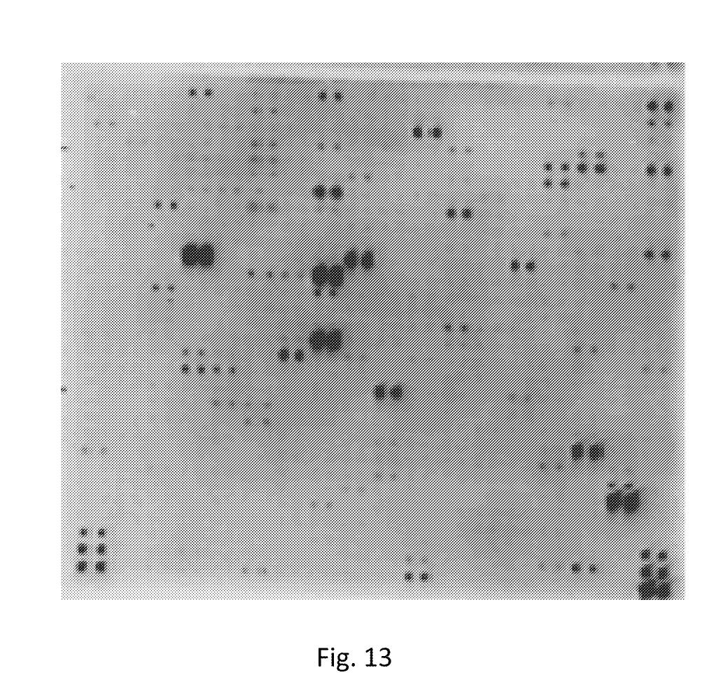

Mesothelial cells were incubated for 24 hours in serum free media and the conditioned media isolated and subjected to analysis using an antibody array (RayBio Cat# AAH-BLM-1-4) that is designed to detect 507 unique proteins (FIG. 13). Secreted molecules discovered in conditioned media from omentum mesothelial cells by this analysis include Activin C (INHBC), AXL receptor tyrosine kinase (AXL), Cancer Antigen 125 (MUC16), Carbohydrate Antigen 19-9, chemokine C-C motif ligand 28 (CCL28), CD30 Ligand (TNFSF8), cysteine rich transmembrane bone morphogenic regulator 1 (CRIM1), c-src tyrosine kinase (CSK), Decorin (DCN), dickkopf homolog 1 (Dkk1), Ectodysplasin A (EDA), epidermal growth factor receptor (EGFR), aminoacyl tRNA synthetase complex-interacting multifunctional protein 1 (AIMP1), chemokine C-X-C motif ligand 5 (CXCL5), Endostatin (COL18A1), Endothelin (EDN1), PANDER (FAM3B), fibroblast growth factor 2 (FGF2), fibroblast growth factor 11 (FGF11), fibroblast growth factor 16 (FGF16), fibroblast growth factor 7 (FGF7), Follistatin (FST), Follistatin-like 1 (FSTL1), Galectin-3 (LGALS3), colony stimulating factor 3 (CSF3), growth differentiation factor 3 (GDF3), growth differentiation factor 5 (GDF5), growth differentiation factor 9 (GDF9), Glypican 3 (GPC3), Glypican 5 (GPC5), GREMLIN (GREM1), chemokine C-X-C motif ligand 1 (CXCL1), intercellular adhesion molecule 1 (ICAM1), insulin-like growth factor binding protein 2 (IGFBP2), insulin-like growth factor binding protein 3 (IGFBP3), insulin-like growth factor binding protein 6 (IGFBP-6), insulin-like growth factor binding protein 7 (IGFBP7), insulin-like growth factor 2 receptor (IGF2 R), interleukin 1 alpha (IL1A), interleukin 1 family member 6 (IL1F6), interleukin 1 family member 9 (IL1F9), interleukin 15 receptor alpha (IL15RA), interleukin 17 (IL17A), interleukin 25 (IL25), interleukin 23 (IL23), interleukin 3 (IL3), interleukin 4 (IL4), interleukin 6 (IL6), interleukin 7 (IL7), interleukin 8 (IL8), interleukin 9 (IL9), kringle containing transmembrane protein 2 (Kremen-2), lipoprotein (LPA), low density lipoprotein receptor-related protein 1 (LRP1), low density lipoprotein receptor-related protein 6 (LRP6), chemokine C-C motif ligand 2 (CCL2), colony stimulating factor 1 (CSF1), chemokine C-X-C motif ligand 2 (CXCL2), matrix metallopeptidase 1 (MMP1), matrix metallopeptidase 10 (MMP10), matrix metallopeptidase 11 (MMP11), neuregulin 3 (NRG3), oncostatin M (OSM), tumor necrosis factor receptor superfamily member 11b (TNFRSF11B), pregnancy-associated plasma protein A (PAPPA), pentraxin 3 (PTX3), granulin (GRN), chemokine C-X-C motif ligand 12 (CXCL12), secreted frizzled-related protein 4 (SFRP4), interleukin 6 signal transducer and soluble interleukin 6 signal transducer (IL6ST), synaptotagmin-like 1 (STYLI), SMAD family member 4 (SMAD4), secreted protein acidic cysteine-rich (SPARC), tissue factor pathway inhibitor (TFPI), Thrombospondin\thrombospondin 1 (THBS1), TIMP metallopeptidase inhibitor 1 (TIMP1), TIMP metallopeptidase inhibitor 2 (TIMP2), tumor necrosis factor receptor superfamily member 1A (TNFRSF1A), vasorin (VASN), vascular cell adhesion molecule 1 (VCAM1), vascular endothelial growth factor A (VEGFA), vascular endothelial growth factor C (VEGFC), ectodysplasin A2 receptor (EDA2R), visfatin (NAMPT), omentin (ITLN1).

### Example 15

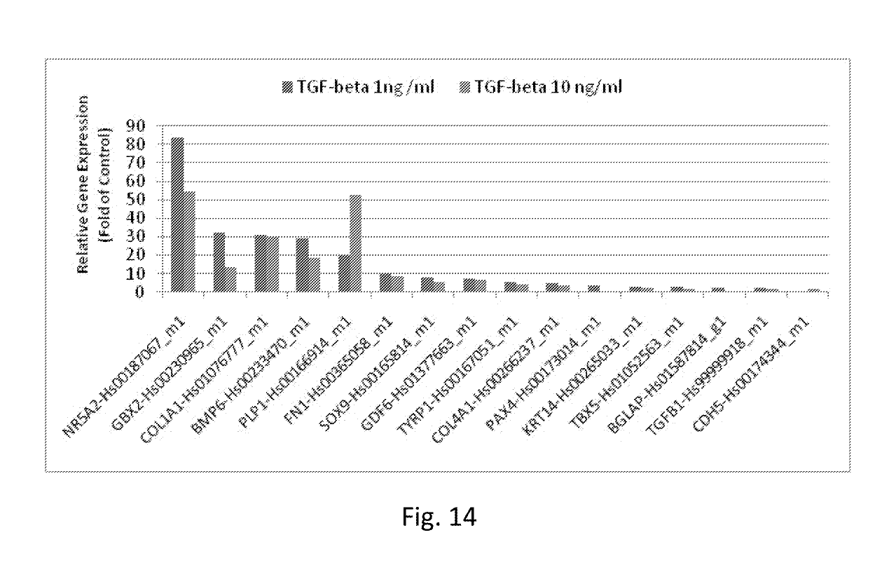

Omental mesothelial cells were induced to undergo epithelial to mesenchymal transition by treatment with transforming growth factor-β (1 ng/ml or 10 ng/ml) for 72 hours. Total RNA was isolated and the levels of mRNA encoding NR5A2 determined by quantitative real-time PCR (FIG. 14).

What is claimed is:

1. A method of providing a source of omentum mesothelial cells comprising providing a substantially pure isolated population of human omentum mesothelial cells and using the cells or any part of the cells thereof in an assay for a selected drug under development.

2. A method according to claim 1 wherein the cells are omentum cells.

3. A method according to claim 1 wherein the cells are myocardial cells.

4. A method according to claim 1 wherein the cells are renal cells.

5. A method to claim 1 wherein the cells are liver cells.

6. A method to claim 1 wherein the cells are pancreas cells.

7. A method of claim 1 wherein the cells are intestinal cells.

8. A method of claim 1 wherein the cells are peritoneal cells.

9. A method according to claim 1 wherein the cells or part thereof are used to monitor a particular cell process.

10. A method according to claim 9 where in the cell process is selected from the group comprising cell growth, cell death, and activation or inhibition of a cellular process.

11. A method of providing a source of proteins for a bioassay comprising isolating proteins from an isolated population of human omentum, myocardial, liver, lung, renal, peritoneal, intestinal or pancreatic mesothelial cells and using the proteins as at least one component in the bioassay.

12. A method according to claim 11 wherein the bioassay is designed to assay a disease from the group comprising obesity, type 2 diabetes, fatty liver disease and cardiovascular disease.

13. A method according to claim 11 wherein the isolated protein is a secreted protein selected from the group comprising:
Activin C (INHBC), AXL receptor tyrosine kinase (AXL), Cancer Antigen 125 (MUC16), Carbohydrate Antigen 19-9, chemokine C-C motif ligand 28 (CCL28), CD30 Ligand (TNFSF8), cysteine rich transmembrane bone morphogenic regulator 1 (CRIM1), c-src tyrosine kinase (CSK), Decorin (DCN), dickkopf homolog 1(Dkk1), Ectodysplasin A (EDA), epidermal growth factor receptor (EGFR), aminoacyl tRNA synthetase complex-interacting multifunctional protein 1 (AIMP1), chemokine C-X-C motif ligand 5 (CXCL5), Endostatin (COL18A1), Endothelin (EDN1), PANDER (FAM3B), fibroblast growth factor 2 (FGF2), fibroblast growth factor 11 (FGF11), fibroblast growth factor 16 (FGF16), fibroblast growth factor 7 (FGF7), Follistatin (FST), Follistatin-like 1 (FSTL1), Galectin-3 (LGALS3), colony stimulating factor 3 (CSF3), growth differentiation factor 3 (GDF3), growth differentiation factor 5 (GDF5), growth differentiation factor 9 (GDF9), Glypican 3 (GPC3), Glypican 5 (GPC5), GREMLIN (GREM1), chemokine C-X-C motif ligand 1 (CXCL1), intercellular adhesion molecule 1 (ICAM1), insulin-like growth factor binding protein 2 (IGFBP2), insulin-like growth factor binding protein 3 (IGFBP3), insulin-like growth factor binding protein 6 (IGFBP-6), insulin-like growth factor binding protein 7 (IGFBP7), insulin-like growth factor 2 receptor (IGF2 R), interleukin 1 alpha (IL1A), interleukin 1 family member 6 (IL1F6), interleukin 1 family member 9 (IL1F9), interleukin 15 receptor alpha (IL15RA), interleukin 17 (IL17A), interleukin 25 (IL25), interleukin 23 (IL23), interleukin 3 (IL3), interleukin 4 (IL4), interleukin 6 (IL6), interleukin 7 (IL7), interleukin 8 (IL8), interleukin 9 (IL9), kringle containing transmembrane protein 2 (Kremen-2), lipoprotein (LPA), low density lipoprotein receptor-related protein 1 (LRP1), low density lipoprotein receptor-related protein 6 (LRP6), chemokine C-C motif ligand 2 (CCL2), colony stimulating factor 1 (CSF1), chemokine C-X-C motif ligand 2 (CXCL2), matrix metallopeptidase 1 (MMP1), matrix metallopeptidase 10 (MMP10), matrix metallopeptidase 11 (MMP11), neuregulin 3 (NRG3), oncostatin M (OSM), tumor necrosis factor receptor superfamily member 11b (TNFRSF11B), pregnancy-associated plasma protein A (PAPPA), pentraxin 3 (PTX3), granulin (GRN), chemokine C-X-C motif ligand 12 (CXCL12), secreted frizzled-related protein 4 (SFRP4), interleukin 6 signal transducer and soluble interleukin 6 signal transducer (IL6ST), synaptotagmin-like 1 (STYLI), SMAD family member 4 (SMAD4), secreted protein acidic cysteine-rich (SPARC), tissue factor pathway inhibitor (TFPI), Thrombospondin\thrombospondin 1 (THBS1), TIMP metallopeptidase inhibitor 1 (TIMP1), TIMP metallopeptidase inhibitor 2 (TIMP2), tumor necrosis factor receptor superfamily member 1A (TNFRSF1A), vasorin (VASN), vascular cell adhesion molecule 1 (VCAM1), vascular endothelial growth factor A (VEGFA), vascular endothelial growth factor C (VEGFC), ectodysplasin A2 receptor (EDA2R), visfatin (NAMPT), omentin (ITLN1), mesothelin (MSLN).

14. A method according to claim 11 wherein the cells are omentum cells.

15. A method according to claim 11 wherein the cells are myocardial cells

16. A method according to claim 11 wherein the cells are renal cells.

17. A method to claim 11 wherein the cells are liver cells.

18. A method to claim 11 wherein the cells are pancreas cells.

19. A method of claim 11 wherein the cells are intestinal.

20. A method of claim 11 wherein the cells are peritoneal.

21. A method of determining the effect of mesothelial cell proteins on adipocytes comprising co-culturing isolated human omentum, myocardial, liver, lung, renal, peritoneal, intestinal or pancreatic mesothelial cells with human adipocytes.

22. A method of growing soft tissue or organ tissue of a human comprising using human omental mesothelial cells as support cells while growing the human tissue in vitro or ex vivo.

23. A method of isolating mesothelial cells comprising:
a) isolating tissue containing mesothelial cells;
b) treating the tissue with an enzyme solution that separates mesothelial cells from other cell types;
c) removing the tissue from the solution; and
d) centrifuging the solution to isolate the mesothelial cells.

24. A method according to claim 23 wherein the enzyme is a trypsin.

25. A method according to claim 23 wherein the mesothelial cells are omental, myocardial, liver, lung, renal, peritoneal, intestinal or pancreatic mesothelial cells.

26. A method of providing a source of omentum, myocardial, liver, lung, renal, peritoneal, intestinal or pancreatic or mesothelial cells comprising providing a substantially pure isolated population of human omentum, myocardial, liver, lung, renal, peritoneal, intestinal or pancreatic mesothelial cells and using the cells in a selected bioassay.

27. A method of providing a source of omentum, myocardial, liver, lung, renal, peritoneal, intestinal or pancreatic mesothelial cells comprising providing a substantially pure isolated population of human omentum, myocardial, liver, lung, renal, peritoneal, intestinal or pancreatic mesothelial cells and using at least a portion of the cells in a selected cell therapy.

28. A method of providing a source of omentum, myocardial, liver, lung, renal, peritoneal, intestinal or pancreatic mesothelial cells comprising providing a substantially pure isolated population of human omentum, myocardial, liver, lung, renal, peritoneal, intestinal or pancreatic mesothelial cells and using the cells to co-culture with another selected cell and then observing the effect on the selected cell.

29. A method of providing a source of omentum mesothelial cells comprising providing a substantially pure isolated population of human omentum mesothelial cells, and isolation of the conditioned medium from the substantially pure population of omentum mesothelial cells, and using the conditioned medium to treat another selected cell, and then observing the effect on the selected cell.
